(19) United States
(12) Patent Application Publication
(10) Pub. No.: US 2013/0011494 A1
(43) Pub. Date: Jan. 10, 2013

(54) COMPOSITION FOR TREATING DECUBITUS ULCERS IN DIABETIC AND NON-DIABETIC PATIENTS

(76) Inventor: Mohammad Rahimi, Tehran (IR)

(21) Appl. No.: 13/179,533

(22) Filed: Jul. 10, 2011

(51) Int. Cl. A61K 33/30; A61P 17/02

(52) U.S. Cl. 424/642; 424/641

(57) ABSTRACT

The various embodiments herein disclose a composition in the form of an ointment for treating decubitus ulcers in diabetic and non diabetic patients. The composition comprises of therapeutic agents, zinc oxide, vegetable oils and powdered talc. The therapeutic agents include salts of nystatin, tetracycline and neomycin, respectively. The vegetable oils include an olive oil, a soya oil and a sweet almond oil. The composition is effective in treatment of bed sore ulcers or decubitus ulcers in, both diabetic and non-diabetic patients. A method is also provided for preparing a composition in the form of an ointment for treating decubitus ulcers.

### BACKGROUND

1. Technical field

The embodiments herein generally relate to the field of decubitus ulcers and particularly to the treatment of decubitus ulcers. The embodiments herein more particularly relate to a composition for treatment of decubitus ulcers in both diabetic and non-diabetic patients and a method for preparing the composition.

2. Description of the Related Art

Bedsores, more properly known as pressure ulcers or decubitus ulcers, are lesions caused by many factors such as: unrelieved pressure; friction; humidity; shearing forces; temperature; age; continence and medication; to any part of the body, especially portions over bony or cartilaginous areas such as sacrum, elbows, knees, and ankles. Although often prevented and treatable if found early, they can be very difficult to prevent in frail elderly patients, wheel chair users and terminally ill patients. Bedsores are often fatal—even under the auspices of medical care—and are one of the leading iatrogenic causes of death reported in developed countries, second only to adverse drug reactions.

Bed sores are a localized area of tissue injury that develops when soft tissue is compressed between a bony prominence and an external surface for a prolonged period of time. The external surface may be a mattress, a chair or wheelchair, or even other parts of the body. The soft tissues of the body, such as skin and muscle, depend upon blood vessels to carry nutrients to the tissues, and to remove waste products. Bed sores result when prolonged pressure prevents sufficient blood flow to supply the tissues with nutrients. The resulting bed sore represents the death of the involved soft tissues. The primary cure and treatment is to remove the pressure by turning the patient regularly i.e. every two hours. But if the ulcers are very deep, then it is required to provide some extra medications to the patients for early recovery. Ointments with medicaments are used by applying them directly on the skin with ulcers.

There have been many prior arts that suggest ointments for bedsore treatment. But none of the ointment composition disclosed in the prior art ointment is effective in the treatment of injuries, in addition to bed sore ulcers. The injuries may include injuries in diabetic and non-diabetic people, infectious or noninfectious sores, burns, varicose ulcers, old wounds and sunburns.

Hence there is need for a new composition that treats bedsore or decubitus ulcers in addition to injuries for both diabetic and non-diabetic patients. There is also a need for a composition that treats infectious or noninfectious sore, burns, varicose ulcers, old wounds and sunburns.

The above mentioned shortcomings, disadvantages and problems are addressed herein and which will be understood by reading and studying the following specification.

### OBJECTIVES OF THE EMBODIMENTS

The primary object of the embodiments herein is to provide a composition for the treatment of injuries, infectious or noninfectious sores, burns, varicose ulcers, old wounds and sunburns, in addition to a treatment of bed sore or decubitus ulcers.

Another object of the embodiments herein is to provide a composition that is effective for both diabetic and non-diabetic patients.

These and other objects and advantages of the embodiments herein will become readily apparent from the following detailed description taken in conjunction with the accompanying drawings.

### SUMMARY

The various embodiments herein provide a composition for treating decubitus ulcers or bed sore in diabetic and non-diabetic patients. The composition further comprises a predetermined amount of a plurality of therapeutic agents, a base, a plurality of vegetable oils, an antiseptic and a protective agent and powdered talc, respectively. The plurality of therapeutic agents includes salts of nystatin, salts of tetracycline, salts of neomycin, and combination thereof The salt of neomycin is a sulfate salt. The base is petrolatum. The plurality of vegetable oils includes an olive oil, soya oil, sweet almond oil and a combination thereof The vegetable oil comprises 10.5% of the whole composition. The antiseptic and the protective agent is zinc oxide.

According to one embodiment herein, the composition is in the form of an ointment, i.e. for external use only, and prevents the formation of ulcer.

According to one embodiment herein, the ointment treats a bed sore ulcer or a decubitus ulcer by or within at least a month. The composition in the embodiments herein is effective in both, diabetic and non-diabetic patients. The ointment is also effective in sun burns, scalding and fire burns. The ointment in the embodiments herein, is free from any side effects.

According to one embodiment herein, the ointment is kept under 30 degrees centigrade. Freezing is to be avoided for more effect. When applied on any wounded area or an ulcer site, the ointment forms a layer with a thickness of 2 or 3 millimeters. The composition releases the medicament or the therapeutic agent in 24 hours.

According to an embodiment herein, a composition for treatment of bed sore ulcers or decubitus ulcers comprises of following ingredients: talc powdered, zinc oxide, olive oil, soya oil, nystatin, tetracycline, neomycin (as sulfate), petrolatum, sweet almond oil. The ointment is for external use only. The ointment disclosed in the embodiments prevents the formation of decubitus ulcers. The ointment is be rubbed regularly on the red and livid part of the body that is vulnerable and wounded by decubitus ulcer. The ointment also recovers a superficially placed decubitus ulcer. The ointment recovers a more developed and deeper ulcer by a month or more, depending on the depth and development of the ulcer.

The ointment in the embodiments is absolutely effective in diabetic patients. If the ulcer is infected, the ointment relieves the infection and usually there is no need for taking or injection of any other antibiotic. However, if the ulcer and infection is highly developed, antibiotic is also taken or injected. In the case of scalding and fire burn, the ointment is thoroughly efficient. Also the ointment is useful for treatment of sun burns.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating the various steps in a method of preparing a composition for treating decubitus ulcer in diabetic and non-diabetic patients, according to one embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The embodiments herein provide a composition for treatment of decubitus ulcers or bed sore ulcers for both diabetic and non-diabetic patients. The composition is in the form of an ointment. The embodiments herein also provide a method of preparing the ointment.

The composition comprises a predetermined amount of a plurality of therapeutic agents, a base, a plurality of vegetable oils, an antiseptic and a protective agent and powdered talc, respectively. The plurality of therapeutic agents includes a nystatin, a tetracycline, a neomycin and a salt and combination thereof The neomycin is in the form of a sulfate. The base is petrolatum. The plurality of vegetable oils includes an olive oil, soya oil, sweet almond oil and a combination thereof The vegetable oil is in a quantity of 10.5% of the whole composition. The antiseptic and the protective agent is zinc oxide. The composition is in the form of an ointment, for external use only, and prevents the formation of ulcer. The ointment treats a bed sore ulcer or a decubitus ulcer by or within at least a month. The composition in the embodiments herein is effective in both, diabetic and non-diabetic patients. The ointment is also effective in sun burns, scalding and fire burns. The ointment in the embodiments herein, is free from any side effects. The ointment is kept under 30 degrees centigrade. Freezing is to be avoided for enhancing the effect of treatment. When applied, the ointment forms a layer over a wound site or wound area, with a thickness of 2 or 3 millimeters. The composition releases the medicament or the therapeutic agent in 24 hours.

According to an embodiment herein, a composition for treatment of bed sore ulcers or decubitus ulcers comprises the following ingredients: powdered talc, zinc oxide, olive oil, soya oil, nystatin, tetracycline, neomycin (as sulfate), petrolatum, sweet almond oil. The composition is in the form of an ointment. The ointment disclosed in the embodiments herein prevents the formation of decubitus ulcers. The ointment is rubbed regularly on the red and livid part of the body that is vulnerably wounded by decubitus ulcer. The ointment also recovers a superficially placed decubitus ulcer. The ointment recovers a more developed and deeper ulcer by a month or more, depending on the depth and development of the ulcer. The ointment disclosed in the embodiments herein is absolutely effective in diabetic patients. If the ulcer is infected, the ointment relieves the infection and usually there is no need for taking or injection of any other antibiotic. However, if the ulcer and infection is highly developed, antibiotic is also taken or injected. In the case of scalding and fire burn, the ointment is thoroughly efficient. Also the ointment is useful for treatment of sun burns.

According to one embodiment herein, the method of preparing the ointment comprises the steps of mixing a predetermined amount of a plurality of therapeutic agent, a predetermined amount of an antiseptic and a protective agent, a predetermined amount of powdered talc with a predetermined amount of plurality of mixed vegetable oil to form a mixture. The plurality of therapeutic agent is selected from a group comprising of a nystatin, a tetracycline, a neomycin, a salt and a combination thereof The antiseptic and the protective agent herein is zinc oxide. The plurality of vegetable oil is selected from a group comprising of olive oil, soya oil, sweet almond oil and a combination thereof Further, the mixture is stirred until it becomes homogenous. The base is stirred separately for some time and added to the already formed and stirred mixture. The base herein is petrolatum. The base is stirred and mixed until an evenly distributed composition in the form of an ointment is obtained.

According to an embodiment, a method is provided for preparing a composition for treating decubitus ulcer in diabetic and non-diabetic patients. The method involves mixing a predetermined amount of a plurality of vegetable oils with a predetermined amount of a plurality of therapeutic agents. Then a predetermined amount of an antiseptic and a protective agent is added. Further a predetermined amount of a powdered talc is added while continuing the mixing process to form a mixture. The mixture is stirring until the mixture becomes homogenous. A base element is stirred separately and adding to the homogenous mixture while continuing the stirring process to form an evenly distributed mixture. The mixture is in the form of an ointment.

The plurality of vegetable oil is selected from a group comprising of an olive oil, soya oil, sweet almond oil and a combination thereof The plurality of therapeutic agent is selected from a group comprising of a salt of nystatin, a salt of tetracycline, a salt of neomycin and a combination thereof The antiseptic and the protective agent is zinc oxide. The base is petrolatum and Vaseline. The predetermined amount the plurality of vegetable oil is 10.5%. The neomycin is in the form of a sulfate. The composition treats the decubitus ulcer by at least a month and wherein the composition releases the therapeutic agent within 24 hours. The composition is effective in treating a sun burn. The composition is effective in treating a scald. The composition is effective in treating a fire burn. The composition is kept under 30 degrees centigrade and a freezing of the composition is avoided.

According to an embodiment herein, a method of making the ointment involves mixing zinc oxide, therapeutic agents and powdered talc with oils to form a mixture. The mixture is then stirred continuously until it becomes homogenous. The petrolatum is stirred separately. The mixture is added to the petrolatum while stirring and mixing is further continued until a homogenous mixture is obtained in the form of an ointment.

The vegetable oils comprise of 10.5% of the composition. The three types of oil include an olive oil, a soya oil and a sweet almond oil. The preservative used herein is within a limit specified according to the medical pharmacopoeia. The vegetable oils have a high level of amino acids and fats. The high level of amino acids and fats softens the wound site and provides more adequate regulation of blood flow. The tissues are fed better with the medicament and the recovery process is accelerated.

The preservatives are applied within the limit to increase drug stability period.

Vaseline or petrolatum is added for completing the 100 grams of ointment formula. Vaseline used herein has a softening effect and is used as a base for the effective medication formula. As Vaseline extricates the effective materials slowly, it prevents auto-oxidation of the ointment materials.

The solid materials, such as, zinc oxide have some properties such as antiseptic, protective and sedative effects.

The therapeutic agents according to the embodiments herein, are the antibiotics, wherein the antibiotics or therapeutic agents have influences on a wide continuum of infections and gram-positive and gram-negative bacteria. Antibiotics include salts of nystatin, salts of tetracycline, neomycin (as sulfate). According to one embodiment herein, the nystatin is applied for skin fungus and skin infections.

The ointment should be kept under 30 degrees centigrade and freezing should be avoided to be effective in the treatment of the injuries. The injuries include injuries in diabetic and non-diabetic people, infectious or noninfectious sore, burns, varicose ulcers, old wounds and sunburns.

The ointment disclosed in the embodiments herein is free from side effects as shown by the consequences of clinical trials in the hospital of the university.

The ointment disclosed in the embodiments herein, forms a cover or layer with a thickness of 2 or 3 millimeters over the wound site. The bandage of the wound causes the active substance of the medicine to release gradually to recover the infectious and non-infectious ulcers. The maximum time for the release of medication is 24 hours.

The bandage for the superficial infectious decubitus ulcer should be changed once every 24 hours. But for a deeper infectious ulcer, in which the epidermis or dermis of the skin is wounded, the bandage should be changed once in every 12 hours or twice a day. The thickness of the cover or layer of the ointment should be about 2 or 3 millimeters.

In the case of deep ulcers, the ointment is to be rubbed on a piece of sterile gauze pad and then be kept inside the wound site in a way that the ointment is placed on the ulcer and thoroughly covers it. The wound cavity is again thoroughly filled with the sterile gauze so that the wound cavity is at the same level to the unwounded skin. Then the wound is be bandaged.

The amounts and ranges presented for 100 grams of the formula are the most preferred amounts.

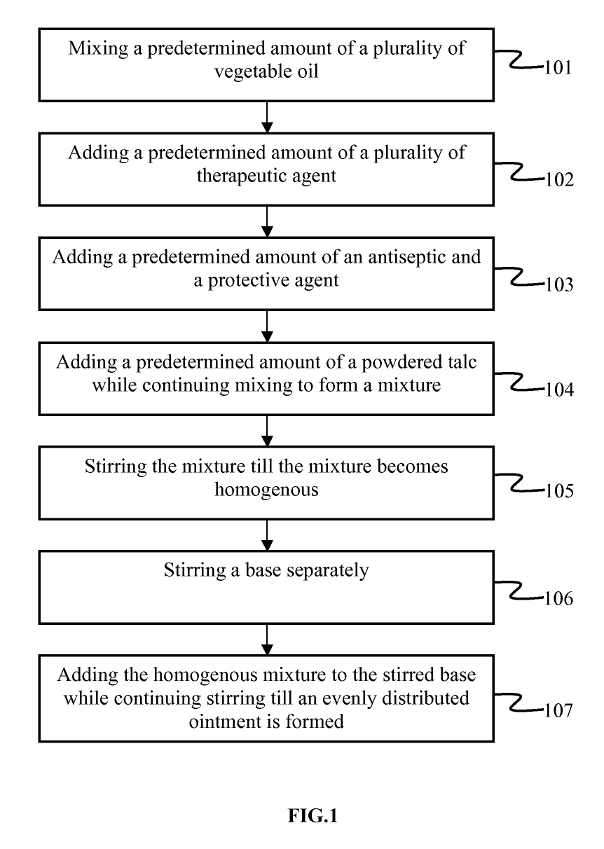

FIG. 1 is a flow chart showing the various steps in a method preparing a composition for treating decubitus ulcer in diabetic and non-diabetic patients, according to one embodiment herein. With respect to FIG. 1, a predetermined amount of a plurality of vegetable oil is mixed (101). The plurality of vegetable oil is selected from a group comprising of an olive oil, soya oil, sweet almond oil and a combination thereof The predetermined amount the plurality of vegetable oil is 10.5% of the whole composition. A predetermined amount of a plurality of therapeutic agent is added (102). The plurality of therapeutic agent is selected from a group comprising of a salt of nystatin, a salt of tetracycline, a salt of neomycin and a combination thereof The neomycin is in the form of a sulfate. A predetermined amount of an antiseptic and a protective agent is added (103). The antiseptic and the protective agent is zinc oxide. A predetermined amount of powdered talc is added while continuing the mixing process to form a mixture (104). The mixture is stirred till it becomes homogenous (105). Further, a base is stirred separately (106). The base is petrolatum and Vaseline. The homogenous mixture is added to the stirred base while continuing the stirring process to form an evenly distributed mixture in the form of an ointment (107). The composition treats the decubitus ulcer by at least a month and wherein the composition releases the therapeutic agent within 24 hours. The composition is effective in treating a sun burn, scald, fire burn. The composition is kept under 30 degrees centigrade and a freezing of the composition is avoided.

### Experimental Data

### EXAMPLE 1

In clinical experiments implemented in the hospital of the university, 58 patients went under treatment in 14 months. Out of them, 46 were men and 12 were women and all were suffering from bed sore. From which, 11 patients were suffering with diabetes. The total number of patients suffering from wounds with the depth of 3 or 4 millimeters was 48. The area of wound was between 5 to 7 square centimeters. The wounds recovered without any side effect during 7 to 10 days.

Ten patients with deep ulcers were then discharged from the hospital with the relative recovery of about 80 to 90 percent. Rest of the treatment was done at home. The therapeutic effects of the ointment herein, or BP-85, in terms of short term treatment and recovery of the ulcers without side effects were approved in diabetic and non-diabetic patients.

### EXAMPLE 2

Evaluation of ointment effects on antimicrobial factors of the ulcer in experimental and non-experimental conditions: On the category of staphylococcus, Pseudomonas, Enterobacteriaceae, the 10% dilution of ointment inhibited the microbial growth thoroughly and their number decreased to zero after 48 hours.

On some types of bacteria such as: gram-negative anaerobic bacillus, ointment in a pure mode was completely effective. The bacteria were removed after 48 hours.

### EXAMPLE 3

Evaluation of the effects through causing infectious ulcer on animals: This experiment was done on four categories of binary group of laboratory rabbits and also four categories of binary group of white mice. The injection involved intra-dermal injection of disease producing microbes in the back of the animal (with the diameter of 5 centimeters). The ulcer was caused through local pressure (ischemia & anoxia). After three days, the ulcer was covered with a layer having a thickness of 2 millimeters of the ointment applied once a day and this action was continued for 7 days.

Results show that the erythema of the ulcer was removed in epidermis and dermis and was recovered. Erythema is a redness of the skin, caused by hyperemia of the capillaries in the lower layers of the skin. It occurs with any skin injury, infection, or inflammation. All the animals had full recovery after 6 days and created ulcer was fully recovered. Therefore, clinical trial is recommended for evaluation.

### EXAMPLE 4

Currently, for the treatment of infectious ulcers, parenteral and oral antibiotics are prescribed besides the prescribed ointments (which has only antibiotic combinations). However, by using the ointment described in the embodiments herein, or BP-85, in the experiments, the infection of the ulcer was removed needless of parenteral and oral antibiotics, and the ulcer site was recovered in a short time.

The embodiments herein provide a composition in the form of an ointment that is to be applied externally, for the treatment of decubitus ulcers. The composition also treats infectious wounds, scars, fire burns, sun burns, scalds etc. The composition treats ulcers irrespective of a diabetic nature of a patient i.e. the composition are effective for both diabetic and non-diabetic patients.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A composition for treating decubitus ulcer in diabetic and non-diabetic patients comprising:
a predetermined amount of at least one therapeutic agent, wherein the at least one therapeutic agent is selected from a group comprising of a salt of nystatin, a salt of tetracycline, a salt of neomycin and a combination thereof;
a predetermined amount of a base, wherein the base is petrolatum and vaseline;
a predetermined amount of at least one vegetable oil, wherein the at least one vegetable oil is selected from a group comprising of an olive oil, a soya oil, a sweet almond oil and a combination thereof;
an antiseptic and protective agent, wherein the antiseptic and protective agent is zinc oxide; and
a powdered talc.

2. The composition according to claim 1, wherein the composition is in a form of an ointment.

3. The composition according to claim 2, wherein the ointment is applied for external use only.

4. The composition according to claim 1, wherein the predetermined amount of the atleast one vegetable oil comprises 10.5% of the composition.

5. The composition according to claim 1, wherein the neomycin is in the form of a sulfate.

6. The composition according to claim 2, wherein the ointment is applied for treating the decubitus ulcer in diabetic patients and in non-diabetic patients in a month.

7. The composition according to claim 2, wherein the ointment is applied for treating s a sun burn.

8. The composition according to claim 2, wherein the ointment is applied for treating a scald.

9. The composition according to claim 2, wherein the ointment is applied for treating a fire burn.

10. The composition according to claim 2, wherein the ointment is kept under 30 degrees centigrade and prevented from freezing.

11. The composition according to claim 1, wherein the composition releases the therapeutic agent within 24 hours.

12. A method for preparing a composition for treating decubitus ulcer in diabetic and non-diabetic patients comprises:
mixing a predetermined amount of a plurality of vegetable oil, wherein the plurality of vegetable oil is selected from a group comprising of an olive oil, a soya oil, a sweet almond oil and a combination thereof;
adding a predetermined amount of a plurality of therapeutic agent, wherein the plurality of therapeutic agent is selected from a group comprising of a salt of nystatin, a salt of tetracycline, a salt of neomycin and a combination thereof;
adding a predetermined amount of an antiseptic and a protective agent, wherein the antiseptic and the protective agent is zinc oxide;
adding a predetermined amount of a powdered talc while continuing mixing to form a mixture;
stirring the mixture, wherein the mixture is stirred till the mixture becomes homogenous;
stirring a base separately, wherein the base is petrolatum and vaseline;
adding the homogenous mixture to the stirred base while continuing stirring to form an evenly distributed mixture, wherein the mixture is in the form of an ointment.

13. The method according to claim 12, wherein the predetermined amount the plurality of vegetable oil is 10.5%.

14. The method according to claim 12, wherein the neomycin is in the form of a sulfate.

15. The method according to claim 12, wherein the ointment is kept under 30 degrees centigrade and a freezing of the ointment is avoided.
